SEQUENCE LISTING (15 sequences)

SEQ ID NO: 1 - DNA, 1225 bp, Homo sapiens
ccgcagcgccggagtcaaacggttcccggcccagtcccgtcctgcagcagtctgcctcctctttcaacatgacagatgccgctgtgtccttcgccaaggacttcctggcaggtggagtggccgcagccatctccaagacggcggtagcgcccatcgagcgggtcaagctgctgctgcaggtgcagcatgccagcaagcagatcactgcagataagcaatacaaaggcattatagactgcgtggtccgtattcccaaggagcagggagttctgtccttctggcgcggtaacctggccaatgtcatcagatacttccccacccaggctcttaacttcgccttcaaagataaatacaagcagatcttcctgggtggtgtggacaagagaacccagttttggcgctactttgcagggaatctggcatcgggtggtgccgcaggggccacatccctgtgttttgtgtaccctcttgattttgcccgtacccgtctagcagctgatgtgggtaaagctggagctgaaagggaattccgaggcctcggtgactgcctggttaagatctacaaatctgatgggattaagggcctgtaccaaggctttaacgtgtctgtgcagggtattatcatctaccgagccgcctacttcggtatctatgacactgcaaagggaatgcttccggatcccaagaacactcacatcgtcatcagctggatgatcgcacagactgtcactgctgttgccgggttgacttcctatccatttgacaccgttcgccgccgcatgatgatgcagtcagggcgcaaaggaactgacatcatgtacacaggcacgcttgactgctggcggaagattgctcgtgatgaaggaggcaaagcttttttcaagggtgcatggtccaatgttctcagaggcatgggtggtgcttttgtgcttgtcttgtatgatgaaatcaagaagtacacataagttatttcctaggatttttccccctgtgaacaggcatgttgtattctataacacaatcttgagcattcttgacagactcctggctgtcagtttctcagtggcaactactttactggttgaaaatgggaagcaataatattcatctgaccagttttcctctaaagccatttccatgatgatgatgatgggactcaattgtattttttatttcagtcactcctgataaataacaaatttggagaaataaaaatatctaaaat

SEQ ID NO: 2 - RNA, 19 nt, Homo sapiens
gcagaucacugcagauaag

SEQ ID NO: 3 - RNA, 9 nt, Homo sapiens
uucaagaga

SEQ ID NO: 4 - DNA, 20 bp, Homo sapiens
ctgagagcgtcgagctgtca

SEQ ID NO: 5 - DNA, 19 bp, Homo sapiens
ctcaatgaagcatctcttc

SEQ ID NO: 6 - DNA, 19 bp, Homo sapiens
ccgcagcgccgtagtcaaa

SEQ ID NO: 7 - DNA, 20 bp, Homo sapiens
agtctgtcaagaatgctcaa

SEQ ID NO: 8 - DNA, 29 bp, Homo sapiens
gaattcaaatgtctcagagcaaccgggag

SEQ ID NO: 9 - DNA, 30 bp, Homo sapiens
gcggccgcattccgactgaagagtgagccc

SEQ ID NO: 10 - DNA, 16 bp, Homo sapiens
gacgggtccggggagc

SEQ ID NO: 11 - DNA, 20 bp, Homo sapiens
cagcccatcttccagatggt

SEQ ID NO: 12 - DNA, 23 bp, Homo sapiens
ggaaatcgtgcgtgacattaagg

SEQ ID NO: 13 - DNA, 22 bp, Homo sapiens
ggcttttaggatggcaagggac

SEQ ID NO: 14 - RNA, 19 nt, Homo sapiens
cugacaucauguacacagg

SEQ ID NO: 15 - RNA, 19 nt, Homo sapiens
gauugcucgugaugaagga

(19) United States
(12) Patent Application Publication
(10) Pub. No.: US 2009/0202623 A1
(43) Pub. Date: Aug. 13, 2009

(54) GENE THERAPY FOR CANCER USING SMALL INTERFERING RNA SPECIFIC TO ANT2 AND A METHOD TO OVERCOME TOLERANCE TO ANTITUMOR AGENT

(76) Inventors: Chul Woo Kim, Seoul (KR); Ji Young Jang, Seoul (KR)

Correspondence Address: KLARQUIST SPARKMAN, LLP, 121 SW SALMON STREET, SUITE 1600, PORTLAND, OR, 97204, US

(21) Appl. No.: 12/293,416

(22) PCT Filed: Apr. 11, 2007
(86) PCT No.: PCT/KR07/01758

§ 371 Date: Sep. 17, 2008

(30) Foreign Application Priority Data
Apr. 11, 2006 (KR) 10-206-0032823

(51) Int. Cl. A61K 48/00; C07H 21/02; C12N 15/85; A61K 31/7105; A61K 9/127; A61P 35/00

(52) U.S. Cl. 424/450; 536/24.5; 435/320.1

(57) ABSTRACT

The present invention relates to a small interfering RNA (siRNA) suppressing the expression of adenine nucleotide trnaslocator 2 (ANT2) gene and an anticancer agent containing the same. Particularly, the invention relates to ANT2 siRNA comprising a sense sequence selected from the nucleotide sequences of ANT2 mRNA, a hairpin loop sequence and an antisense sequence binding complementarily to the said sense sequence and an anticancer agent containing the same. ANT2 siRNA of the present invention inhibits the expression of ANT2 gene, suggesting that it inhibits the growth of cancer cells exhibiting high level of ANT2. Therefore, ANT2 siRNA of the invention can be effectively used for gene therapy for cancer treatment and further prevents the anticancer effect from decreasing by anticancer drug resistance of cancer cells.

### TECHNICAL FIELD

The present invention relates to a small interfering RNA (siRNA) suppressing the expression of adenine nucleotide trnaslocator 2 (ANT2) gene and an anticancer agent containing the same, more precisely ANT2 siRNA comprising a sense sequence of ANT2 mRNA nucleotide sequence, a hairpin loop sequence and an antisense sequence binding complementarily to the said sense sequence and an anticancer agent containing the same.

### BACKGROUND ART

Tumor is a result of abnormal, incontrollable and disordered cell proliferation including excessive abnormal cell proliferation. When a tumor exhibits destructive proliferation, infiltration and metastasis, it is classified as a malignant tumor. In particular from the view point of molecular biology, a tumor is considered as a genetic disease caused by mutation of a gene.

To treat malignant tumors, three treatment methods which are surgical operation, radiotherapy and chemotherapy have been conducted either separately or together. Particularly, surgical operation is a method to eliminate most of pathogenic tissues, which is thus very effective to remove tumors growing in the breast, colon and skin but not so effective to treat tumors in spine and dispersive tumors.

Radiotherapy has been performed to treat acute inflammatory, benign or malignant tumors, endocrine disorders and allergies, and it has been effective to treat such malignant tumors resulted from abnormal rapid cell division. However, the ratiotherapy carries serious side effects such as functional disorder or defect of normal cells, outbreak of cutaneous disorders on the treated area and particularly retardation and anostosis in children.

Chemotherapy is a method to disturb duplication or metabolism of cancer cells, which has been performed to treat breast cancer, lung cancer and testicular tumor. The biggest problem of this treatment method is the side effect carried by systemic chemotherapy. Side effects of such chemotherapy are lethal and thus increase anxiety and fear for the treatment. One of the representative side effects of chemotherapy is dose limiting toxicity (DLT). Mucositis is one of examples of DLT for various anticancer agents (antimetabolic agents such as 5-fluorouracil and methotrexate, and antitumor antibiotics such as doxorubicin). Most cases of side effects require hospitalization or at least need pain killers. So, side effects by chemotherapy and radiotherapy are serious matters for the treatment of cancer patients.

In the meantime, gene therapy is based on the DNA recombination technique, which is the method to insert a therapeutic gene into cancer patient cells to correct gene defect or to endow a novel functions to disordered cells to treat or prevent various genetic diseases caused by mutations of genes, cancer, cardiovascular diseases, infective diseases, autoimmune diseases, etc. More particularly, gene therapy is a method to treat the said diseases by inducing intracellular expressions of normal proteins or therapeutic target proteins by conveying a therapeutic gene into a target organ. Gene therapy has an excellent selectivity, compared with other treatment methods using drugs and can be applied for a long term with improved treatment effect on difficult diseases. To enhance the therapeutic effect of gene therapy, gene transfer technique is essential for the realization of high efficient gene expression in target cells.

A gene carrier is a mediator for the insertion of a therapeutic gene into a target cell. A preferable gene carrier is the one that is not harmful for human, can be mass-produced and has ability to transmit a therapeutic gene effectively and induce constant expression of the therapeutic gene. Thus, gene transfer technique is a key factor for gene therapy and representative gene carriers most wanted for gene therapy so far are exemplified by viral carriers such as adenovirus, adeno-associated virus (AAV), and retrovirus; and non-viral carriers such as liposome and polyethyleneimine.

It is one of the strategies of gene therapy to control tumor cells by using a tumor suppressor gene, a tumor-specific killer virus, a suicide gene and an immunoregulation gene. Particularly, the method using a tumor suppressor gene is to treat cancer by conveying the original form of a tumor suppressor gene such as p53, which is deficient or mutated in many cancer patients. The method using a tumor-specific killer virus is to treat cancer by conveying a virus gene carrier that can be proliferated selectively in tumor cells into cancer patients by taking advantage of the activity of a tumor suppressor gene transformed in cancer tissues. The basic strategy of the above two methods is to kill tumor cells directly. In the meantime, the method using a suicide gene is to induce suicide of tumor cells by inserting sensitive genes such as HSK-TK. The method using an immunoregulation gene is to treat disease indirectly by stimulating T-cell mediated tumor cell recognition by delivering a gene increasing antitumor immune response such as interleukin 12 (IL12), interleukin 4 (IL4), interleukin 7 (IL7), γ-interferon and tumor necrosis factor (TNF) or by inducing apoptosis by interrupting tumor inducing proteins.

In relation to gene therapy among various attempts to treat cancer, the present inventors selected ANT (adenine nucleotide translocator) as a target gene to develop an effective safe anticancer agent.

ANT (adenine nucleotide tranlocator) is an enzyme found in inner membrane (IM) of mitochondria, which imports ADP from cytoplasm through VDAC (voltage dependent anion channel) of outer membrane (OM) of mitochondria and exports ATP generated in electron transfer chain system into cytoplasm (HLA Vieira, et al., Cell Death and Differentiation, 7, 1146-1154, 2000).

It is also known that ANT playing a key role in energy metabolism of cells is classified into ANT1, ANT2 and ANT3. Particularly ANT2 exhibits low expression rate in normal cells but is highly expressed in cancer cells or similarly highly proliferated cells, which seems to be closely related to glycolysis under anaerobic condition, so that ANT2 is rising up as a new target for cancer treatment (Chevrollier, A, et al., Med. Sci., 21(2), 156-161, 2005). However, the previous report only suggested the possibility of application to cancer treatment and in fact there has been no reports saying that ANT2 is a target gene which is effective for cancer treatment.

It has been disclosed recently that double stranded RNA (dsRNA) inserted in animal or plant cells could decompose mRNA corresponding to the dsRNA and thereby inhibit a specific protein synthesis, which is called ‘RNA interference’ (Sharp, P. A., et al., Genes Dev., 16, 485-490, 2001). At this time, dsRNA is converted into siRNA (small interfering RNA) by an unknown mechanism and decomposes corresponding mRNA. But, when dsRNA having at least 30 nucleotides is used, non-specific reactions might nullify protein synthesis interruption or at least make the interruption inefficient (Hunter, T. et al., J. Biol. Chem., 250, 409-417, 1975; [symbol] Robertson, H. D. and Mathews, M. B., Biochemie., 78, 909-914, 1996). To overcome the above problem, a new technique has been developed to synthesize double stranded siRNA composed of 21 oligomers and to insert the siRNA into mammalian cells to decompose corresponding mRNA to interrupt a specific target protein synthesis (Hutvagner, H. D. et al., Science, 29, 834-838, 2001).

In vivo/in vitro experiments have been vigorously performed as follows in order to treat diseases including cancer by synthesizing double stranded siRNA composed of 21 oligomers. For example, β-catenin that is involved in rapid growth of cancer cells was effectively eliminated from cultured colon cancer cells and mouse colon cancer models by using synthetic β-catenin siRNA (Verma, U. N., et al., Clinical Cancer Res., 9, 1291-1300, 2003; and Annick, H. B., et al., PNAS USA, 99, 14849-14854, 2002).

It was also reported that when multidrug resistance 1 (MDR1) siRNA synthetic oligomer produced to overcome drug resistance of cancer cells, which has been a barrier for chemotherapy, was inserted in MDR1 expressing cells, MDR1 protein synthesis was blocked (Wu, H. et al., Cancer Res., 63, 1515-1519, 2003). When cycline E siRNA synthetic oligomer was treated to cyclin E over-expressing liver cancer cells, the proliferation of cultured liver cancer cells and/or liver cancer cells transplanted into a mouse was suppressed (Kaiyi, L. et al., Cancer Res., 63, 3593-3597, 2003).

The above results indicate that siRNA that is over-expressed in cancer cells and at the same time able to interrupt selectively a protein involved in rapid growth of cancer cells can be developed as an effective anticancer agent. Nevertheless, synthetic siRNA allegedly has disadvantages as follows; synthetic siRNA oligomer requires high costs for its synthesis, exhibits low intracellular transmission rate, induces non-specific reaction that might induce cytotoxicity and has short half-life in vivo which suggests that the effect is not constant and thereby the injection has to be repeated. So, in vivo application of synthetic siRNA is limited.

Both viral and non-viral gene carriers that can express siRNA in cells are expected to overcome the said disadvantages of synthetic siRNA oligomer greatly.

The present inventors observed that ANT2 siRNA that was believed to interrupt ANT2 protein synthesis could effectively inhibited the growth of cancer cells where ANT2 was over-expressed, and completed this invention by confirming that ANT2 siRNA can be used as an anticancer agent.

### Disclosure

### Technical Problem

It is an object of the present invention to provide an anticancer agent that is able to inhibit the proliferation of cancer cells especially over-expressing ANT2 which is closely involved in the development and progress of cancer.

### Technical Solution

To achieve the above object, the present invention provides a small interfering RNA (siRNA) specifically binding to mRNA of adenine nucleotide translocator 2 (ANT2).

The present invention also provides an expression vector containing the polynucleotide corresponding to the siRNA nucleotide sequence.

The present invention further provides a treatment method for cancer containing the step of administering the said siRNA or the said expression vector to an individual with cancer.

The present invention also provides an anticancer composition containing the said siRNA or the said expression vector.

Hereinafter, the present invention is described in detail.

The present invention provides a small interfering RNA (siRNA) specifically binding to mRNA of adenine nucleotide translocator 2 (ANT2).

In this invention, the said siRNA is composed of a 17-25 mer sense sequence, a 7-11 mer hairpin loop sequence and an antisense sequence corresponding to the above sense sequence which is selected from nucleotide sequences of adenine nucleotide translocator 2 (ANT2) mRNA. The sense sequence corresponds to the nucleotide sequence of ANT2 mRNA represented by SEQ. ID. NO: 1 (see FIG. 15) and the sense sequence itself is represented by SEQ. ID. NO: 2. The hairpin loop sequence is preferably represented by SEQ. ID. NO: 3 but not always limited thereto.

The present invention also provides an expression vector containing the polynucleotide corresponding to the siRNA nucleotide sequence.

In this invention, the plasmid expression vector containing the polynucleotide corresponding to the nucleotide sequence of ANT2 siRNA is constructed with five T bases (T₅) that are transcription termination sequences and the polynucleotide corresponding to the nucleotide sequence of ANT2 siRNA designed to form H1 (RNA polymerase III) and hairpin loop structure. The polynucleotide corresponding to the nucleotide sequence of ANT2 siRNA was cloned into Bam H1/Hind III region of H1 promoter is generated by cloning into Bam H1/Hind III region of pSilencer 3.1-H1 puro plasmid vector (Ambion, Austin, Tex.) designed to be expressed by H1 promoter (see FIG. 1 and FIG. 2). However, in this invention, the vector to express ANT2 siRNA is not limited to pSilence 3.1-H1 puro vector and the promoter to express ANT2 siRNA is not limited to H1 promoter, either. For example, Pol III promoter that can start transcription by eukaryotic RNA polymerase III and a promoter such as U6 promoter or CMV promoter that can induce gene expression in mammalian cells are also preferably used but not always limited thereto.

The present inventors confirmed that ANT2 siRNA expression vector treated to cancer cells could induce apoptosis (see FIGS. 4C and 4D) and suppress the proliferation of ANT2 over-expressing breast cancer cell line (MDA-MB-231) (see FIG. 4B).

The present inventors investigated the mechanism of anticancer activity of ANT2 siRNA. As a result, the inventors confirmed that cancer cell death is directly induced by ANT2 siRNA (see FIG. 7 and FIG. 8) and at the same time the anticancer effect of ANT2 siRNA is more effective by indirect inducement of cancer cell death by promoting the expressions of TNF-α and TNFR1 receptor (see FIGS. 7-11).

To investigate the in vivo anticancer effect of ANT2 siRNA, ANT2 siRNA expression vector was inserted into breast cancer cells, which were then injected under the right femoral region of a nude mouse, followed by measurement of time-dependent tumor sizes. As a result, the size of a tumor was reduced in the mouse by the injection of breast cancer cell line where ANT2 siRNA was expressed, compared with that of control (see FIG. 12).

It was further confirmed that MDR (multidrug resistance) of breast cancer cell line (MDA-MB-231) was reduced by the insertion of ANT2 siRNA, the reactivity of an anticancer agent such as gemcitabine was increased and IC₅₀ was also reduced (see FIG. 13 and FIG. 14). The above results indicate that ANT2 siRNA insertion in cancer cells contribute to the overcoming of multidrug resistance and enhancement of anticancer effect with even lower dose of an anticancer agent.

From the above results, it was confirmed that when ANT2 siRNA expression vector of the invention is inserted into ANT2 over-expressing cancer cells, the expression of ANT2 is suppressed, ATP synthesis which is necessary energy for cancer cell proliferation is interrupted, the expressions of TNF-α and its receptor TNFR1 inducing apoptosis are increased, and thereby apoptosis of cancer cells is induced, suggesting that tumor growth is greatly inhibited by inducing apoptosis.

ANT2 is over-expressed in most cancer cells including stomach cancer, lung cancer, hepatoma and ovarian cancer, therefore ANT2 siRNA expression vector of the present invention can be applied to a variety of cancers.

The present invention further provides a treatment method for cancer containing the step of administering siRNA or the expression vector to an individual with cancer and an anticancer composition containing siRNA or the expression vector.

ANT2 siRNA and siRNA expression vector of the present invention can be administered locally or systemically in different forms of compositions prepared by using various carriers, for example hypodermic injection, intramuscular injection or intravenouse injection, etc. It is preferred to administer ANT2 siRNA or siRNA expression vector directly to the lesion or inject intravenously as a form of nano particles or a complex with liposome where ligand that can recognize a cancer cell specific marker is attached inside or outside. In the case that the complex is intravenously injected, the complex or nano particles are circulated through blood vessels and then reach tumor tissues. And then they specifically bind to a marker expressed specifically on cancer cell surface, so that ANT2 siRNA or the said siRNA expression vector can be delivered into the inside of cancer cell to induce ANT2 silence, resulting in anticancer effect. Previously, Iwasaki et al added GFP gene or HSV thymidine kinase gene to the hepatitis virus L antigen containing nano particles, and then injected the complex into hepatoma xenograft animal model. It was resultingly observed that tumor growth was inhibited in the animal model in which GFP gene was expressed specifically in hepatoma cells and HSV thymidine kinase gene was inserted (Iwasaki et al., Cancer Gene Ther., 14(1):74-81, 2007). Peng et al also reported that the in vivo systemic administration of a protein-gene complex comprising Apotin and asialoglycoprotein recognized specifically by asialoglycoprotein receptor, a hepatoma specific marker, reduced cancer cell growth (Peng et al., Cancer Gene Ther., 14(1):66-73, 2007). Grzelinski et al reported that the systemic administration of pleiotrophin specific siRNA and polyethyleneimine (PEI) complex inhibited cancer cell growth significantly in glioblastoma animal model (Grzelinski et al., Hum. Gene Ther., 17(7):751-66, 2006). McNamara et al reported that the administration of cancer cell specific aptamer and siRNA chimera RNA involved in cancer cell survival inhibited tumor cell growth significantly in the prostatic cancer xenograft animal model (McNamara et al., Nat. Biotechnol., 24(8):1005-1015, 2006). The said documents are all listed herein as references. As explained above, ANT2 siRNA or the said siRNA expression vector of the present invention can be effectively used for the treatment of cancer by administering them to an individual with cancer according to the method or pathway described in the said reference. Various cancer specific markers have been known and the one reported by Cho is one example (William Chi-shing Cho, Molecular Cancer, 6:1-9, 2007). A marker specific ligand is preferably a receptor or an antibody against a marker. A nano complex for gene therapy is preferably prepared by mixing ANT2 siRNA or the expression vector of the present invention with liposome, polyethyleneglycol (PEG) and polyethyleneimine.

### DESCRIPTION OF DRAWINGS

The application of the preferred embodiments of the present invention is best understood with reference to the accompanying drawings, wherein:

FIG. 1 is a diagram showing the cleavage map of an expression vector for the expression of adenine nucleotide translocator 2 (ANT2) mRNA specific siRNA (small interfering RNA).

FIG. 2 is a diagram showing the cleavage map of an expression vector for the expression of adenine nucleotide translocator 2 (ANT2) mRNA specific siRNA (small interfering RNA).

FIG. 3A is a diagram showing the result of RT-PCR exhibiting the expressions of ANT1 and ANT2 mRNAs in breast cancer cells (MDA-MB-231) and peripheral blood mononuclear cells (PBMC) and FIG. 3B is a diagram showing the result of RT-PCR, which is that ANT2 siRNA expression vector of the present invention reduces ANT2 mRNA expression in breast cancer cells:

Scramble siRNA: negative control

FIG. 4A is a graph showing that the ANT2 siRNA expression vector of the present invention reduces ATP production in breast cancer cells, and FIG. 4B is a graph showing that the ANT2 siRNA expression vector of the present invention inhibits breast cancer cell proliferation, compared with the control scramble siRNA. FIG. 4C is a graph showing the cell survival rate (%) illustrating apoptosis of cancer cells induced by the ANT2 siRNA expression vector and FIG. 4D is a diagram showing the apoptosis of cancer cells by genome DNA fragmentation.

FIG. 5 is a diagram showing the result of RT-PCR(left) and the result of Western blotting (right) each explaining the changes of the levels of Bcl-xL (apoptosis inhibiting factor) and Bax (apoptosis stimulating factor) mRNAs by the ANT2 siRNA expression vector and the effect of the said vector on protein expressions.

FIG. 6 is a graph showing the destruction of the membrane of mitochondria of breast cancer cells induced by ANT2 siRNA, confirmed by staining with DiOC6.

FIG. 7 is a graph showing the result of FACS by using double staining with Annexin V and propidium iodide(PI) to investigate the anticancer effect of ANT2 siRNA expression vector on breast cancer cells by observing direct apoptosis (upper part) and indirect apoptosis (lower part) after 24 hours from the treatment.

FIG. 8 is a graph showing the result of FACS using double staining with Annexin V and propidium iodide (PI).

Particularly, FACS was performed to investigate the anticancer effect of ANT2 siRNA, ANT2 siRNA-2 and ANT2 siRNA-3 expression vectors on breast cancer cells by observing apoptosis after 48 hours from the treatment.

FIG. 9 is a graph showing the relevance between ANT2 siRNA expression vector and TNF-α produced in cancer cells investigated by FACS.

FIG. 10 is a set of a diagram and a graph each showing the relevance between ANT2 siRNA expression vector and the level of TNF-α receptor 1 (TNFR1) mRNA by RT-PCR, and illustrating the correlation between ANT2 siRNA expression vector and TNFR1 by FACS.

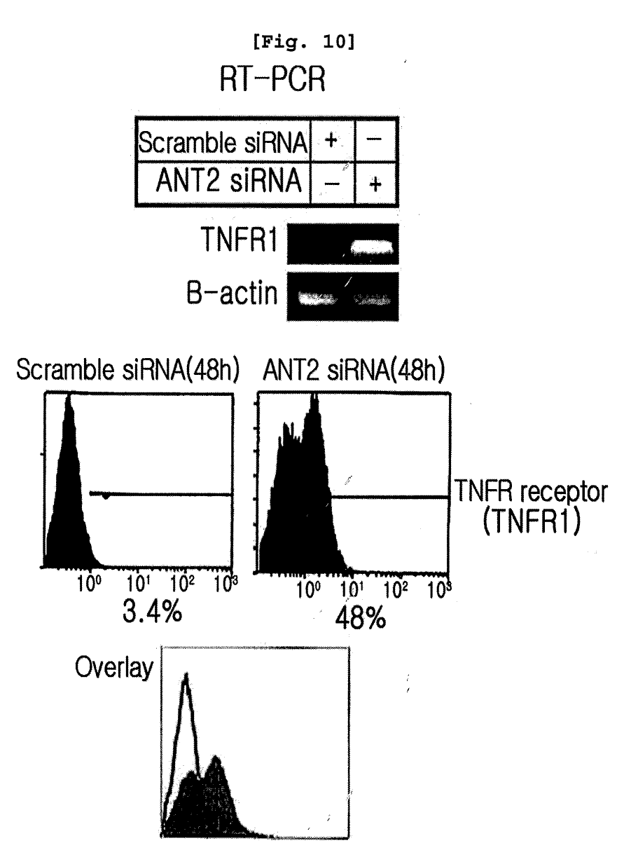

FIG. 11 is a graph showing that whether the increase of TNF-α production in cancer cells by ANT2 siRNA expression vector of the invention could induce cancer cell death indirectly, the medium was neutralized by using TNF-α antibody and the effect on apoptosis was measured by FACS.

FIG. 12A is a graph showing the anticancer effect of ANT2 siRNA or the negative control scramble siRNA expression vector, which was investigated by measuring the size of a tumor in a nude mouse after transplantation of breast cancer cells (MDA-MB-231) containing ANT2 siRNA of the invention or the negative scramble siRNA expression vector under the right femoral region of balb/c nude mouse, and FIG. 12B is a graph showing that ANT2 siRNA of the invention or the negative control scramble siRNA expression vector was introduced into breast cancer cells (MDA-MB-231), which were then transplanted under the right femoral region of balb/c nude mouse, followed by measuring the weight of a tumor separated by dissection on the 60^{th} day from the transplantation.

FIG. 13 is a graph showing the effect of ANT2 siRNA of the present invention on multidrug resistance of breast cancer cells (MDA-MB-231) measured by FACS.

FIG. 14 is a graph showing the association of ANT2 siRNA of the invention with the reactivity of an anticancer agent (gemcitabine) to breast cancer cells (MDA-MB-231).

FIG. 15 is a diagram showing the target sequence of ANR2 siRNA of the invention screened among human ANT2 (Genebank Accession No. NM_{—}001152 and SEQ. ID. NO: 1) nucleotide sequences:

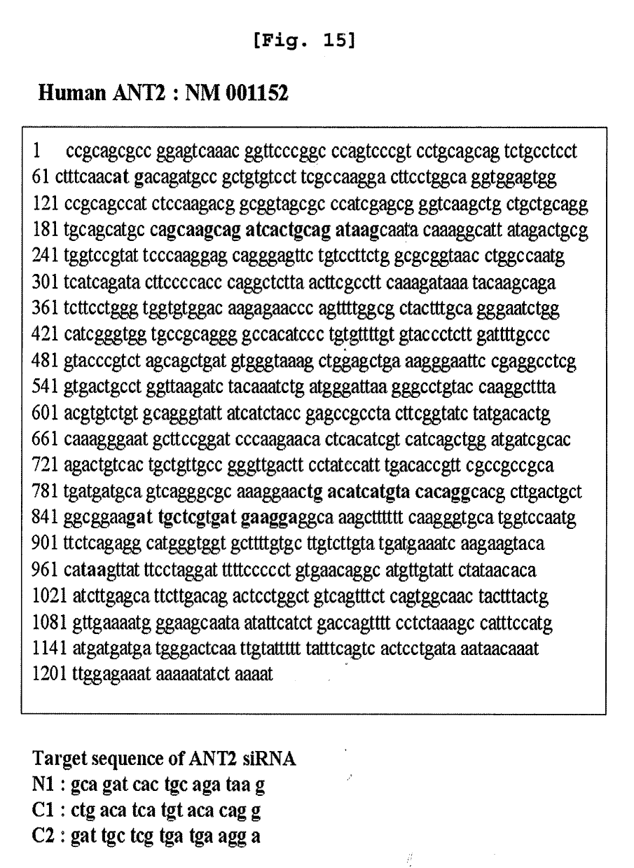

N1: target sequence of ANT2 siRNA;

C1: target sequence of ANT2 siRNA-2; and

C2: target sequence of ANT2 siRNA-3.

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Construction of ANT2 siRNA Expression Vector

ANT2 siRNA was provided by National Center for Biotechnology Information (NCBI, http://www.ncbi.nlm.nih.gov/) and further prepared based on the nucleotide sequence corresponding to the second exon (SEQ. ID. NO: 2) of Genebank Accession No. NM_{—}001152 (SEQ. ID. NO: 1), which is the nucleotide sequence of the most appropriate oligomer of all the candidate sequences obtained from the siRNA prediction program (http://www.ambion.com/technical, resources/siRNA target finder). The present inventors also constructed ANT2 siRNA-2 (SEQ. ID. NO: 14; 5′-CUGACAUCAUGUACACAGG-31) and ANT2 siRNA-3 (SEQ. ID. NO: 15; 5′-GAUUGCUCGUGAUGAAGGA-3′), in addition to ANT2 siRNA for comparison. The construction of ANT2 siRNA, ANT2 siRNA-2 and ANT2 siRNA-3 was conducted by Bionner (Korea).

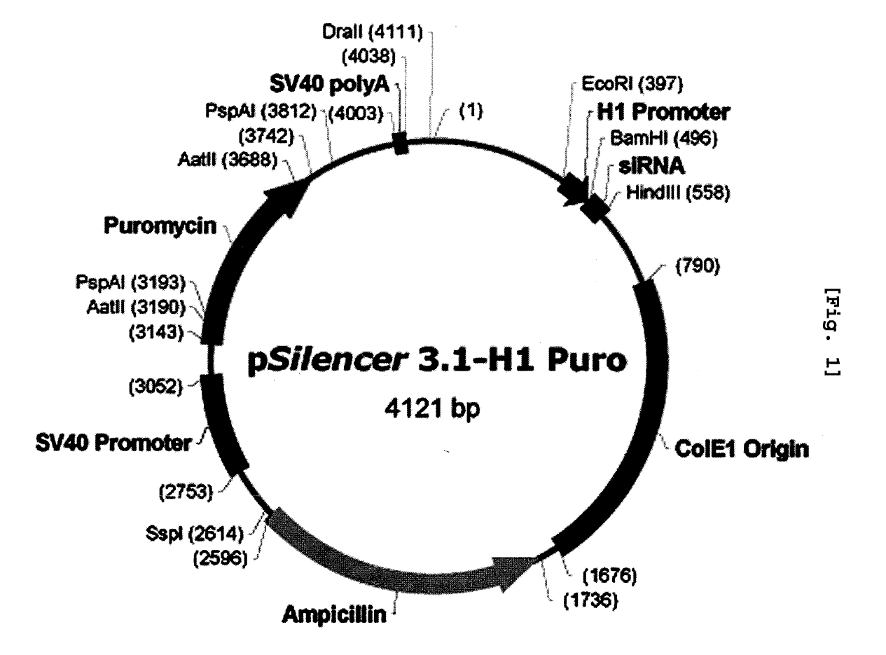

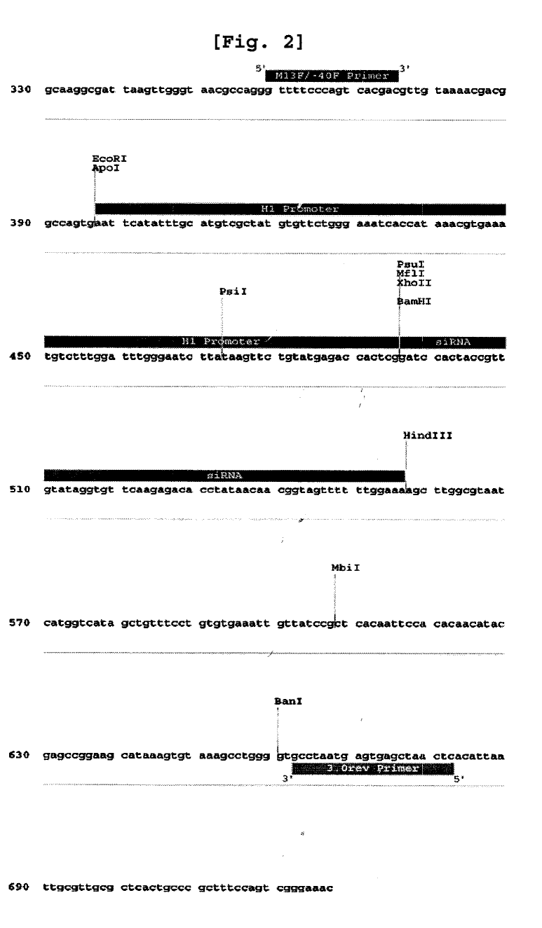

Particularly, the vector was designed to include a sense sequence (5′-GCAGAUCACUGCAGAUAAG-3′, SEQ. ID. NO: 2) corresponding to 197-217 of ANT2 mRNA (SEQ. ID. NO: 1) that is the target sequence of siRNA inhibiting ANT2 expression, a hairpin loop sequence (5′-TTCAAGAGA-3′, SEQ. ID. NO: 3) and an antisense sequence binding complementarily to the said sense sequence. TT was also included in order to increase the expression efficiency of siRNA, which was cloned into Bam HI and Hind III regions of MCS (multi-cloning site) of pSilencer 3.1-H1 puro plasmid vector (Ambion Co.) to be expressed by H1 promoter (FIG. 1 and FIG. 2). In the meantime, scramble siRNA used as a negative control which was not capable of interrupting ANT2 expression but was able to play a same role was purchased from Ambion Co. ANT2 siRNA-2 and ANT2 siRNA-3 were also constructed by the same manner as described above except they were designed to target different sequences.

### Example 2

### Measurement of the Activity of ANT2 siRNA Expression Vector

<2-1> Inhibitory Effect of ANT2 siRNA on ANT2 Expression

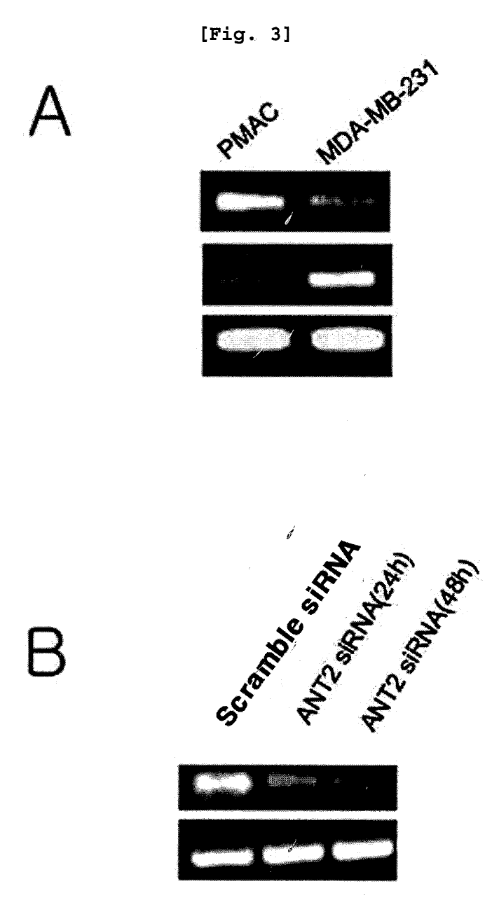

In this invention, ANT2 expressions in different human cancer cell lines were investigated. As a result, the present inventors selected a breast cancer cell line (MDA-MB-231) exhibiting high ANT2 expression for the experiment (FIG. 3A). The MDA-MB-231 cell line of the invention was purchased from Korean Cell Line Bank (KCLB) and cultured in DMEM (Sigma) supplemented with 10% FBS (fetal bovine serum), 100 units/ml of penicillin and 100 ug/ml of streptomycin (Sigma) in a 37° C., 5% CO₂ incubator (Sanyo, Japan).

To investigate whether ANT2 siRNA of the invention could actually inhibit ANT2 expression, RT-PCR was performed with the said breast cancer cell line in the presence of ANT2 siRNA to measure the level of ANT2 expression. Particularly, the cells were distributed into a 6-well plate (2×10⁵ cells) or 100 mm dish (2×10⁶ cells), followed by culture for 24 hours. Then, Lipofectamine 2000 (Invitrogen), pSilencer 3.1-H1 puro ANT2 siRNA vector or pSilencer 3.1-H1 puro scramble siRNA vector was added at the concentration of 2 ug/2×10⁵ cells. Reaction was induced in serum-free medium at room temperature for 15 minutes to let them bind well. The breast cancer cell line was transfected with the reacted medium, followed by further culture for 4 hours. The medium was discarded, and the cells were washed with PBS, followed by further culture for 24-48 hours in serum containing medium.

After 24-48 hours from the transfection, the cells were treated with Trizol (Invitrogen) to separate the total RNA. And cDNA was synthesized from 5 μg of the total RNA by using RT-PCR kit (Promega, Madison, Wis.). The obtained cDNA was denatured at 94° C. for 5 minutes, followed by 35 cycles of denaturation at 94° C. for 1 minute, annealing at 55° C. for 1 minute, polymerization at 72° C. for 2 minutes, and final extension at 72° C. for 5 minutes. PCR product obtained above was electrophoresed on 1% agarose gel to confirm. The primer sequences used for PCR herein are as follows:

1) ANT1: (forward)

(SEQ. ID. NO: 4)

5′-CTG AGA GCG TCG AGC TGT CA-3′;
and
(reverse)

(SEQ. ID. NO: 5)

5′-CTC AAT GAA GCA TCT CTT C-3′;
2) ANT2: (forward)

(SEQ. ID. NO: 6)

5′-CCG CAG CGC CGT AGT CAA A-3′;
and
(reverse)

(SEQ. ID. NO: 7)

5′-AGT CTG TCA AGA ATG CTC AA-3′;
3) Bcl-xL: (forward)

(SEQ. ID. NO: 8)

5′-GAA TTC AAA TGT CTC AGA GCA ACC GGG AG-3′;
and
(reverse)

(SEQ. ID. NO: 9)

5′-GCG GCC GCA TTC CGA CTG AAG AGT GAG CCC-3′;
4) Bax: (forward)

(SEQ. ID. NO: 10)

5′-GAC GGG TCC GGG GAG C-3′;
and
(reverse)

(SEQ. ID. NO: 11)

5′-CAG CCC ATC TTC CAG ATG GT-3′;
5) β-actin: (forward)

(SEQ. ID. NO: 12)

5′-GGA AAT CGT GCG TGA CAT TAA GG-3′;
and
(reverse)

(SEQ. ID. NO: 13)

5′-GGC TTT TAG GAT GGC AAG GGA C-3′.

As explained hereinbefore, expression of ANT2 siRNA was investigated RT-PCR. As a result, from 24 hours after the transfection, intracellular ANT2 mRNA expression was inhibited in MDA-MB-231 cells by ANT2 siRNA and 48 hours later the ANT2 expression was suppressed significantly by ANT2 siRNA (FIG. 3B).

<2-2> Inhibition of ATP Production and Cell Proliferation and Induction of Apoptosis by ANT2 siRNA

The present inventors introduced ANT2 siRNA and the negative control scramble siRNA respectively into the breast cancer cells (MDA-MB-231) which were cultured to investigate ATP production, cell growth inhibition and apoptosis therein.

ATP Production

To measure the level of intracellular ATP, the cells were reacted with CellTiter-Glo™ regent (CellTiter-Glo™ solution and CellTiter-Glo™ substrate, Promega) and then luminescence was measured by using a luminometer (Tecan Instruments) at room temperature.

Cell Growth Inhibition

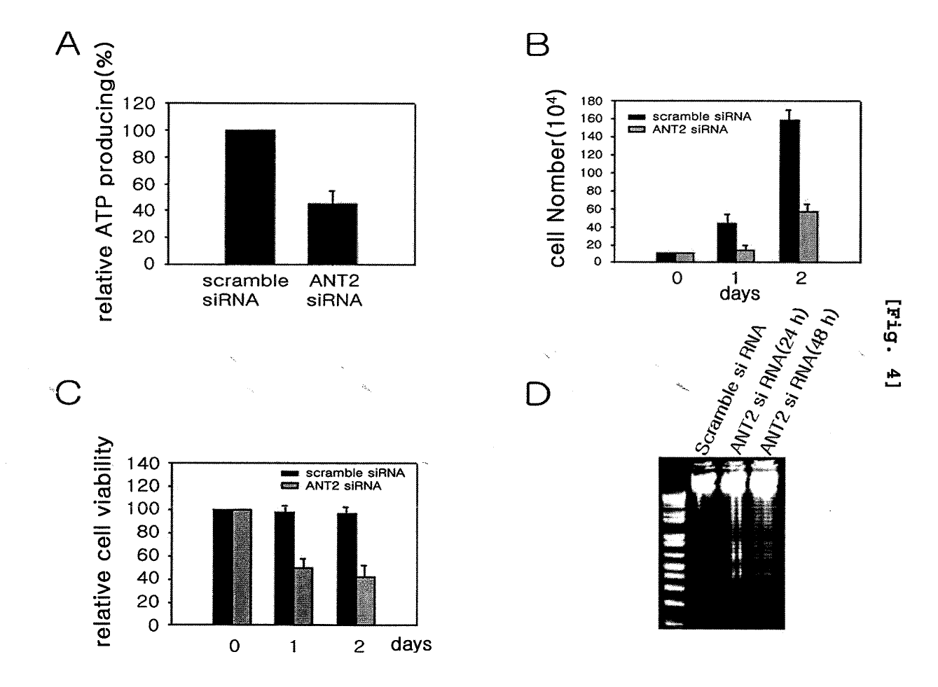

To investigate the effect of ANT2 siRNA on cell growth, MDA-MB-231 cell line was transfected with ANT2 siRNA or scramble siRNA by using Lipofectamine-2000 (Invitrogen) by the same manner as described in the above Example 2. Then the cell number was counted by hemacytometer on the day of transfection, on the next day and two days later (FIG. 4B).

Apoptosis

The transfected cells were reacted with Annexin V and PI (Propidium ionide, BD pharmingen) in a dark room at room temperature for 15 minutes and the cell number was measured by FACS (Epics XL, Coulter, France). Genomic DNA was separated from DNA fragmentation by using genomic DNA kit (Intron, Korea), followed by electrophoresis on 2% agarose gel to measure apoptosis.

As a result, ATP production was reduced in MDA-MB-231 cells transfected with ANT2 siRNA (FIG. 4A), compared with that of the control cells treated with scramble siRNA (FIG. 4A), and cell proliferation was also reduced significantly after the transfection on the next day and two days later as well (FIG. 4B). Regarding apoptosis of MDA-MB-231 cells by ANT2 siRNA, approximately 50% apoptosis effect was observed on the next day of transfection and after two days from the transfection as well (FIG. 4C). DNA fragmentation was significantly observed in the breast cancer cells transfected with ANT2 siRNA, compared with the control cells both on the 24^{th} and 48^{th} hour from the transfection (FIG. 4D).

Therefore, the present inventors confirmed that ANT2 siRNA has an anticancer effect by inducing apoptosis and inhibiting cell proliferation and ATP production specifically associated with ANT2 expression.

<2-3> Regulation of the Expression of Apoptosis Associated Factors by ANT2 siRNA and Destruction of Mitochondria Membrane by ANT2 siRNA

The present inventors observed the expressions of apoptosis associated factors and the changes of mitochondria membrane which are closely associated with apoptosis after insertion of ANT2 siRNA in the breast cancer cell line.

Regulation of the Expressions of Apoptosis Associated Factors

The present inventors transfected MDA-MB-231 cells with ANT2 siRNA or scramble siRNA and cultured thereof. Then the levels of mRNAs of apoptosis associated factors (Bcl-xL; apoptosis inhibiting factor, and Bax; apoptosis inducing factor) were measured by the same manner as described in Example 2.

To investigate protein levels, the cells were transfected with ANT2 siRNA and scramble siRNA respectively, and 48 hours later the cells were recovered, lysed in lysis buffer (5 mmol/L EDTA, 300 mmol/L NaCL, 0.1% 1 gepa, 0.5 mmol/L NaF, 0.5 mmol/L Na₃VO₄, 0.5 mmol/L PMSF, 10 g/mL aprotinin, pepstatin) by using leupeptin (Sigma), and centrifuged (15,000×g, 30 min). The supernatant was obtained to measure protein level by using Brandford solution (Bio-Rad). 50 μg of the protein proceeded to 10% SDS-polyacrylamide gel for electrohporesis, transferred onto polyvinylidene difluoride membrane (Millipore), treated with an antibody (anti-Bcl-xL, anti-Bax, and anti-a tublin (Santa Cruz Biotech) and colored by chemiluminescence detection system (Amersham Pharmacia Biotech).

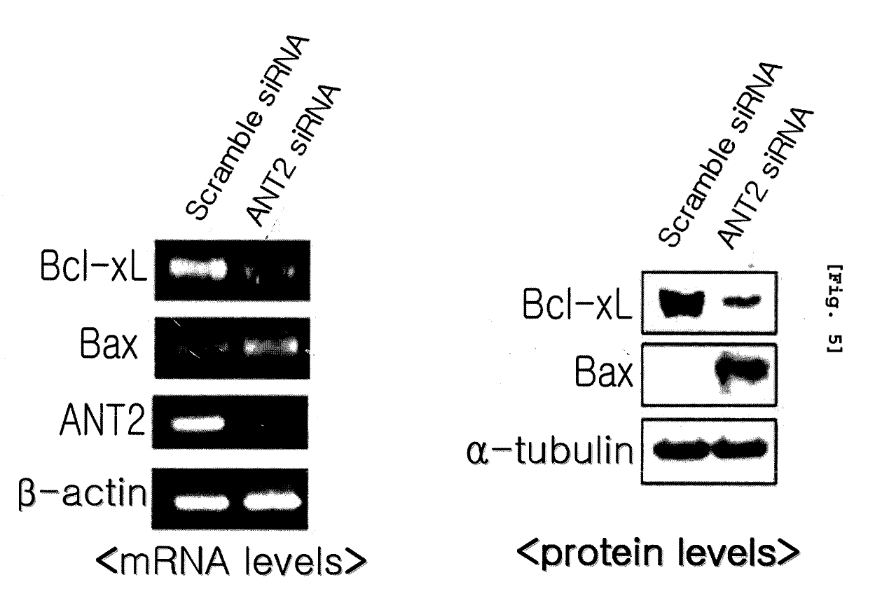

As a result, the levels of Bcl-xL mRNA (apoptosis inhibiting factor) and protein were decreased in cells transfected with ANT2 siRNA and at the same time the levels of Bax mRNA (apoptosis inducing factor) and protein were significantly increased (FIG. 5).

Deconstruction of Mitochondria Membrane

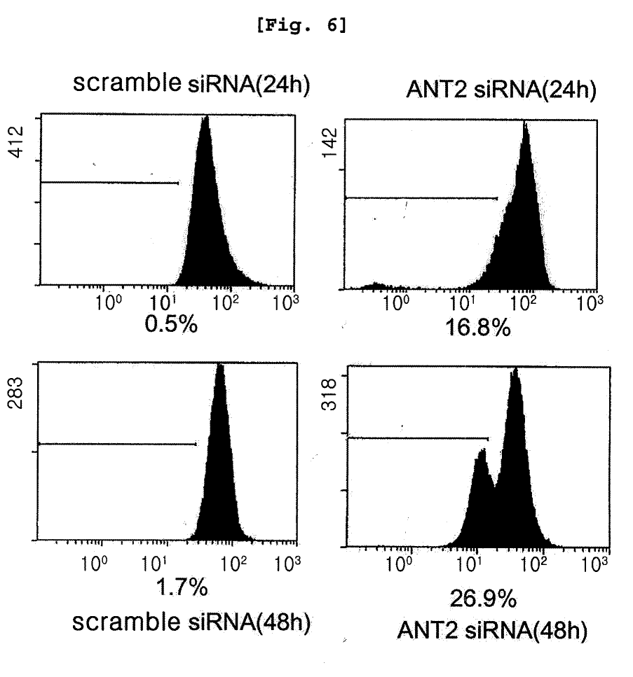

The present inventors investigated deconstruction of mitochondria membrane by ANT2 siRNA by using DiOC6 that can penetrate into the mitochondria membrane. Particularly, to measure deconstruction of membranes of mitochondria of breast cancer cells transfected with ANT2 siRNA, the cells were treated with 20 nM of DiOC6 (Molecular Probes, Eugene, USA), followed by reaction at 37° C. for 15 minutes. As a result, deconstruction of mitochondria membrane was significant in the cells transfected with ANT2 siRNA, compared with the control cells transfected with scramble siRNA, both 24 hours (0.5% vs. 16.8%) and 48 hours (1.7% vs. 26.9%) later (FIG. 6).

The above results indicate that ANT2 siRNA of the present invention induces apoptosis of cancer cells by deconstructing mitochondria membrane associated closely with apoptosis and by regulating the expressions of apoptosis associated factors.

<2-4> Direct and Indirect Effect of Inducing Apoptosis by ANT2 siRNA

To investigate whether apoptosis could induced directly by ANT2 siRNA, the present inventors performed staining with propidium iodide (PI) and Annexin V.

Particularly, the cells were transfected respectively with ANT2 siRNA and scramble siRNA, followed by culture for 24-48 hours. The cells were washed with PBS, to which PI and Annexin V were added. After reaction at room temperature for 15 minutes, OD₄₈₈ was measured by FACS.

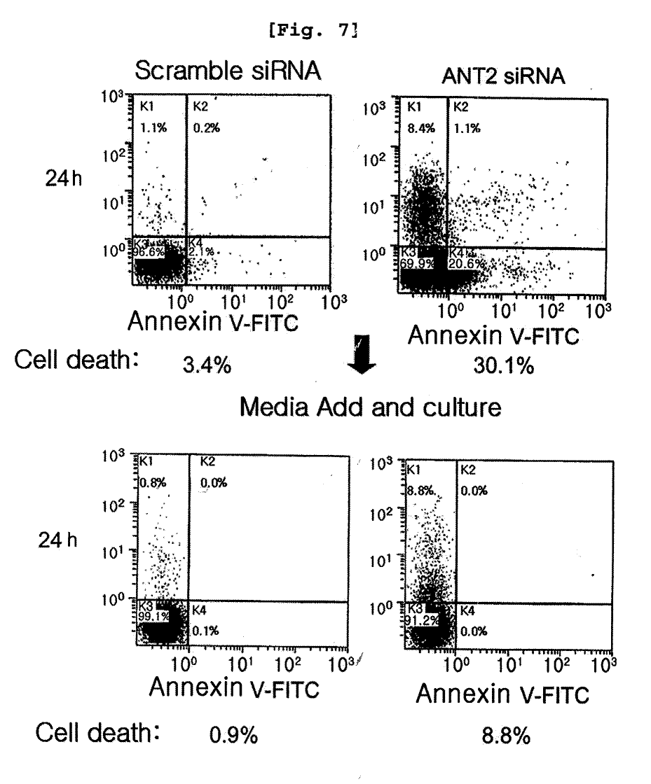

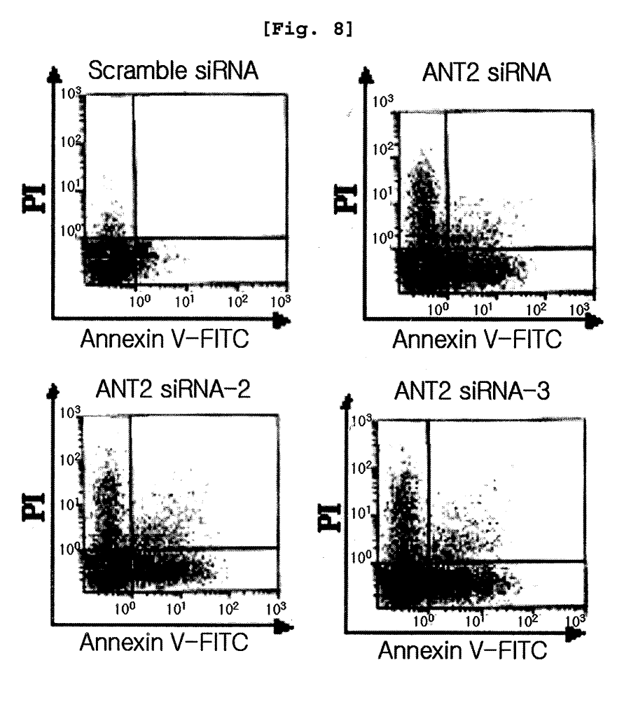

As a result, apoptosis was directly induced by ANT siRNA after 24 (scramble siRNA: 2.4% vs. ANT2 siRNA: 30.1%) and 48 (scramble siRNA: 4.7% vs. ANT2 siRNA: 52.9%) hours from the transfection, compared with the control cells transfected with scramble siRNA. The results observed after 48 hours from the transfection were all consistent among ANT2 siRNA, ANT2 siRNA-2 and ANT2 siRNA-3, and in particular apoptosis was most significantly induced in ANT2 siRNA treated group (FIG. 7 and FIG. 8).

The present inventors further investigated whether ANT2 siRNA could induce apoptosis indirectly, in addition to its direct effect on apoptosis.

Particularly, breast cancer cells were transfected with ANT2 siRNA and scramble siRNA respectively and cultured for 48 hours. Centrifugation was performed to remove cells remaining in medium. Then, the medium was treated to the cells untransfected with the said siRNA, followed by culture for 24 and 48 hours. Apoptosis was observed.

As a result, even though this indirect apoptosis inducing effect was not as high as the direct effect, apoptosis was still induced comparatively high in the cells transfected with ANT2 siRNA, compared with the control cells transfected with scramble siRNA after 24 (control: 0.9% vs. ANT2 siRNA: 8.8%) and 48 (control: 1.8% vs. ANT2 siRNA: 13.2%) hours from the transfection (FIG. 7). The above results indicate that apoptosis is induced indirectly not by ANT siRNA itself but by TNF-α generated in those cells transfected with ANT2 siRNA and thus the cancer treatment effect might be increased by using ANT2 siRNA.

### Example 3

### Mechanism of Inducing Apoptosis by ANT2 siRNA

After observing the indirect apoptosis inducing effect of ANT2 siRNA, the present inventors tried to analyze the mechanism of inducing apoptosis. Particularly, the inventors investigated the expressions of TNF-α and one of its receptors TNF-α receptor 1(TNFR1) in the cancer cell line after ANT2 siRNA treatment. More specifically, ANT2 siRNA and scramble siRNA (control) were introduced into MDA-MB-231 cells, followed by culture for 24 hours. Then, the cells were treated with 10 μg/ml of BFA (brefeldin A: eBioscience, USA) for 6 hours to interrupt the extracellular secretion of TNF-α. Then, the levels of TNF-α and TNFR1 were measured by RT-PCR or FACS.

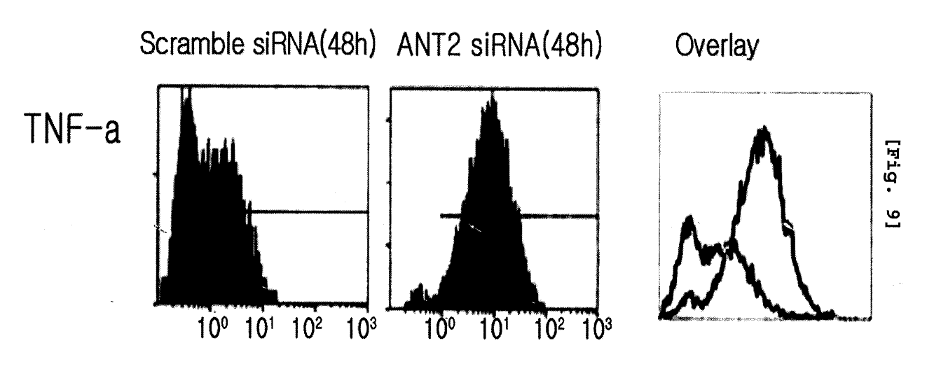

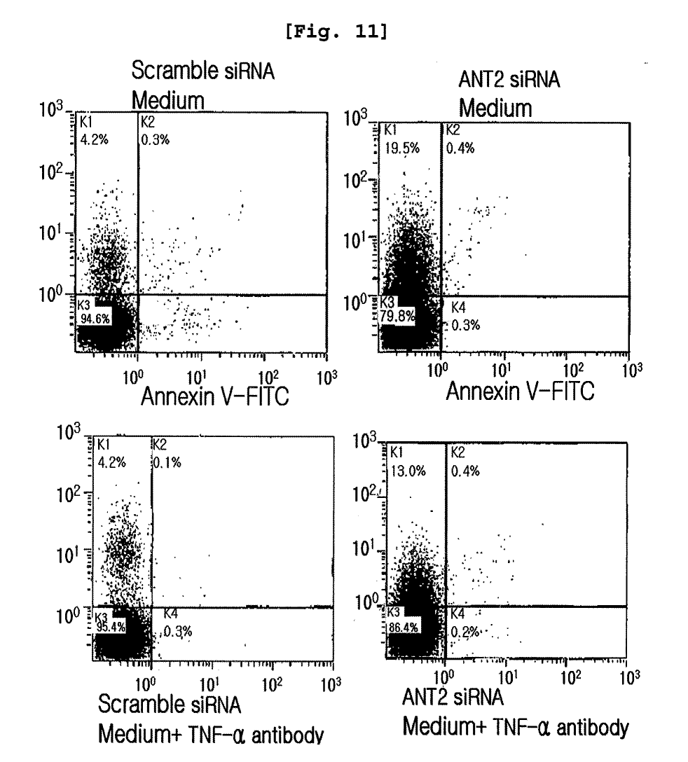

As a result, RT-PCR and FACS analysis confirmed that the levels of TNF-α and TNF-α receptor 1 (TNFR1) significantly increased by ANT2 siRNA in the cells. To confirm whether indirect apoptosis inducing effect was caused by TNF-α or not, the culture medium of cells transfected with ANT2 siRNA or scramble siRNA was neutralized by using TNF-α antibody, which proceeded to cell culture. As a result, the apoptosis inducing effect was reduced, suggesting that TNF-α was involved in indirect apoptosis by ANT2 siRNA. There must be another factors involved in apoptosis since the effect of TNF-a on apoptosis was partial (FIG. 9 and FIG. 11). So, in addition to the direct apoptosis inducing effect of ANT2 siRNA, the increase of the levels of TNF-α and its receptor TNFR1 can enhance the cancer treatment effect. According to the recent reports saying that the direct injection of TNF-α DNA to cancer cells or direct insertion of soluble TNF-α receptor to cancer cells can enhance the cancer treatment effect, the treatment method for cancer using ANT2 siRNA is considered to be very effective.

### Example 4

### Analysis of In Vivo Anticancer Effect of ANT2 siRNA

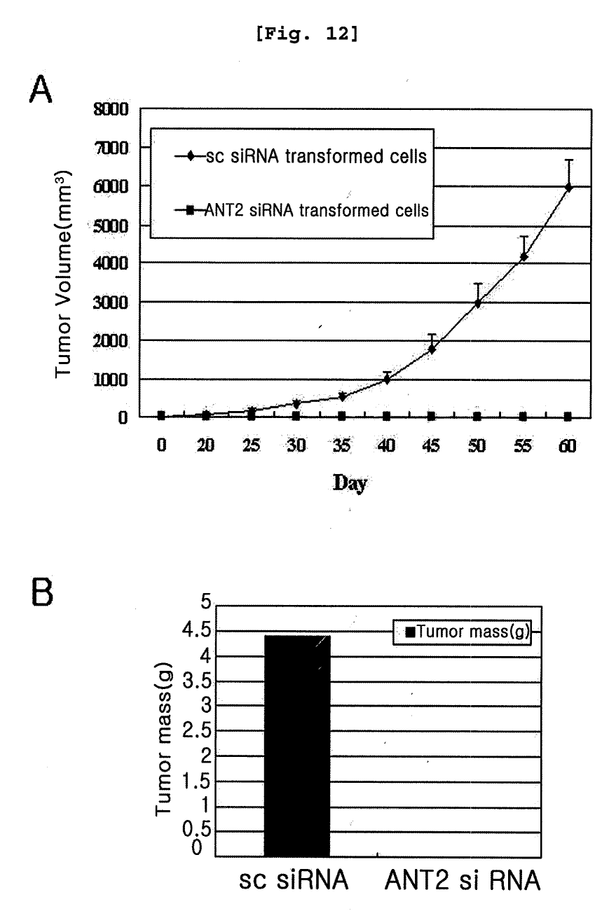

In Example 2, it was observed that the treatment of ANT2 siRNA significantly inhibited breast cancer cell proliferation. To investigate whether this result was consistent with that of in vivo experiment, the present inventors introduced ANT2 siRNA and scramble siRNA into MDA-MB-231 cells (5×10⁶/100 μl). The transfected MDA-MB-231 cells were transplanted under the right femoral of balb/c nuce mice (Charles River Japan, Japan), 5 mice per group, and then the tumor size was observed for 33 days to investigate whether the growth of a tumor generated therein could be inhibited by ANT2 siRNA (FIG. 12). The tumor size was calculated by the following Mathematical Formula 1.

Tumor Volume (mm²)=Minor Axis²×Major Axis×0.523 <Mathematical Formula 1>

As a result, the normal tumor growth was observed in the nude mice transplanted with breast cancer cells transfected with scramble siRNA, whereas the tumor growth was not observed in the nude mice transplanted with breast cancers transfected with ANT2 siRNA. The above result indicates ANT2 siRNA can reduce tumor cell growth significantly still in vivo. The nude mice were dissected on the 60^{th} day of transplantation to measure the weight of a tumor (FIG. 12).

### Example 5

### Reducing Effect of ANT2 siRNA on Anticancer Drug Resistance

To investigate how ANT2 siRNA affects the anticancer drug resistance of cancer cells, the present inventors performed Rho123 staining. The anticancer drug resistance is shown when efflux pump on the cell surface pumps an anticancer drug out of cells and thus the amount of the drug remaining in cells becomes so small. Therefore, the effect of ANT2 siRNA on the anticancer drug resistance can be measured by investigating the activity of efflux pumps on cell surface after the administration of ANT2 siRNA.

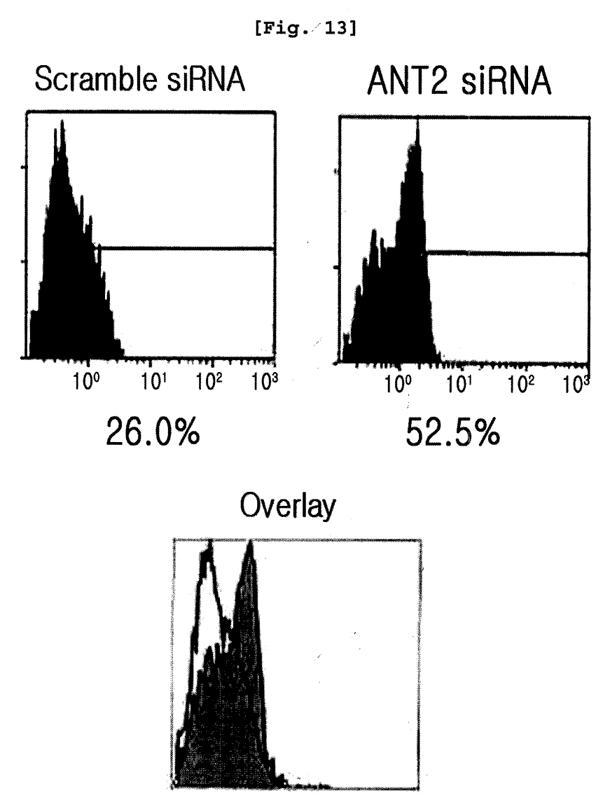

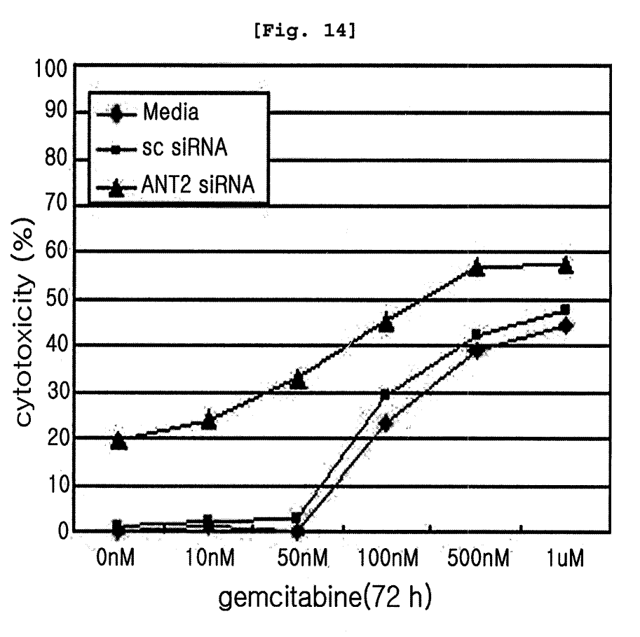

Particularly, to measure the efflux activity, 100 nM of Rhodamine 123 (Sigma) was added to MDA-MB-231 cells (2×10⁵), followed by reaction at 37° C. for 60 minutes. Twenty four hours after the addition, the accumulation of intracellular Rhodamine 123 was increased in the cells transfected with ANT2 siRNA, compared with the cells transfected with scramble siRNA. The above result indicates that ANT2 siRNA reduces the activity of efflux pumps on cell surface, which is associated with anticancer drug resistance of cancer cells. Besides, it was also observed that the reactivity against such anticancer drug as gemcitabine was also increased to reduce IC₅₀ (FIG. 13 and FIG. 14). Therefore, it was confirmed that gene therapy using siRNA can overcome the anticancer drug resistance of cancer cells, minimize the side effects of anticancer drugs by lowering the dosage and thereby increases the treatment effect.

### INDUSTRIAL APPLICABILITY

The present invention relates to gene therapy for cancer using small interfering RNA (siRNA) specifically binding to adenine nucleotide translocator 2 (ANT2). The ANT2 siRNA containing expression vector induces directly or indirectly the decrease of ATP production necessary for tumor cell growth and the increase of TNF-α and its receptor productions involved in apoptosis. Therefore, the expression vector can significantly suppress tumor growth in mouse models transplanted with cultured cancer cells exhibiting high level of ANT2. In conclusion, the expression vector containing ANT2 siRNA can be effectively used for gene therapy for cancer independently or together with other cancer treatment methods.

### Sequence List Text

SEQ. ID. NO: 1 is the polynucleotide sequence of ANT2 gene.

SEQ. ID. NO: 2 is the polynucleotide sequence of ANT2 siRNA.

SEQ. ID. NO: 3 is the polynucleotide sequence of ANT2 hairpin loop.

SEQ. ID. NO: 4 is the polynucleotide sequence of a forward primer for the amplification of ANT1 gene.

SEQ. ID. NO: 5 is the polynucleotide sequence of a reverse primer for the amplification of ANT1 gene.

SEQ. ID. NO: 6 is the polynucleotide sequence of a forward primer for the amplification of ANT2 gene.

SEQ. ID. NO: 7 is the polynucleotide sequence of a reverse primer for the amplification of ANT2 gene.

SEQ. ID. NO: 8 is the polynucleotide sequence of a forward primer for the amplification of Bcl-xL gene.

SEQ. ID. NO: 9 is the polynucleotide sequence of a reverse primer for the amplification of Bcl-xL gene.

SEQ. ID. NO: 10 is the polynucleotide sequence of a forward primer for the amplification of Bax gene.

SEQ. ID. NO: 11 is the polynucleotide sequence of a reverse primer for the amplification of Bax gene.

SEQ. ID. NO: 12 is the polynucleotide sequence of a forward primer for the amplification of M-actin gene.

SEQ. ID. NO: 13 is the polynucleotide sequence of a reverse primer for the amplification of M-actin gene.

SEQ. ID. NO: 14 is the polynucleotide sequence of ANT2 siRNA-2.

SEQ. ID. NO: 15 is the polynucleotide sequence of ANT2 siRNA-3.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A small interfering RNA (siRNA) specifically binding to adenine nucleotide translocator 2 (ANT2) mRNA.

2. The siRNA according to claim 1, which contains a 17-25 mer sense sequence selected from the nucleotide sequence of ANT2 mRNA represented by SEQ. ID. NO: 1.

3. The siRNA according to claim 2, which comprises the sense sequence, a 7-11 mer hairpin loop sequence and an antisense sequence binding complementarily to the sense sequence.

4. The siRNA according to claim 2, wherein the sense sequence is selected from a group consisting of sequences represented by SEQ. ID. NO: 2, NO: 14 and NO: 15.

5. The siRNA according to claim 2 wherein the hairpin loop sequence is the sequence represented by SEQ. ID. NO: 3.

6. An expression vector that expresses the polynucleotide corresponding to the nucleotide sequence of the siRNA of claim 1.

7. The expression vector according to claim 6, which comprises a promoter, ANT2 siRNA designed to form a hairpin loop structure, and a transcription termination signal T₅.

8. The expression vector according to claim 7, wherein the promoter is a Pol III promoter that is able to start transcription by eukaryotic RNA polymerase III.

9. The expression vector according to claim 8, wherein the Pol III promoter is a H1 or U6 promoter.

10. The expression vector according to claim 6, which comprises is pSilencer 3.1-H1 puro that expresses ANT2 siRNA.

11. A treatment method for cancer comprising the step of administering to an individual with cancer the siRNA of claim 1 or an expression vector that expresses the siRNA.

12. The treatment method for cancer according to claim 11, wherein the siRNA or the expression vector forms a nano complex with a carrier.

13. The treatment method for cancer according to claim 12, wherein the carrier comprises a liposome, polyethyleneglycol or polyethyleneimine.

14. The treatment method for cancer according to claim 13, wherein the nano complex additionally comprises a ligand that specifically binds big to a cancer specific marker.

15. The treatment method for cancer according to claim 14, wherein the ligand is bound to the carrier by a covalent bond.

16. An anticancer composition comprising as an effective ingredient the siRNA of claim 1 or an expression vector that expressed the siRNA.

17. The composition according to claim 16, which additionally comprises a pharmaceutically acceptable carrier.

18. The composition according to claim 17, wherein the carrier comprises a liposome, polyethyleneglycol or polyethyleneimine.

19. The composition according to claim 18, which additionally comprises a ligand that specifically binds to a cancer specific marker.

20. The composition according to claim 19, wherein the ligand is bound to the carrier by a covalent bond.
